(19) United States
(12) Patent Application Publication
(10) Pub. No.: US 2014/0303473 A1
(43) Pub. Date: Oct. 9, 2014

(54) OBJECT INFORMATION ACQUIRING APPARATUS AND CONTROL METHOD THEREOF

(71) Applicant: CANON KABUSHIKI KAISHA, TOKYO (JP)

(72) Inventor: Ryuichi Nanaumi, Kyoto-shi (JP)

(73) Assignee: CANON KABUSHIKI KAISHA, TOKYO (JP)

(21) Appl. No.: 14/230,163

(22) Filed: Mar. 31, 2014

(30) Foreign Application Priority Data
Apr. 4, 2013 (JP) 2013-078827

(51) Int. Cl. A61B 5/00

(52) U.S. Cl. CPC A61B 5/0091; A61B 5/0095
USPC 600/407

(57) ABSTRACT

An object information acquiring apparatus, comprises an irradiation unit configured to irradiate light from a light source onto a breast, which is an object; a detection unit configured to detect a signal propagating from the breast by the light irradiated from the irradiation unit; a calculation unit configured to calculate an optical characteristic inside the breast based on the signal detected by the detection unit, wherein the irradiation unit configured to irradiate the light onto the breast in a direction away from a chest wall located at the base of the breast.

### BACKGROUND OF THE INVENTION

1. Field of the Invention

The present invention relates to an object information acquiring apparatus and a control method thereof.

2. Description of the Related Art

In the technical area of biological diagnosis, diagnosis using near infrared light whose absorption by an organism is relatively low has been attempted. In near infrared light diagnosis, spectroscopic information about the tissue of the organism can be acquired, and functional information (e.g. composition ratio, density analysis) of the constituents of an organism based on the spectroscopic information can be acquired. Further, unlike X-ray imaging or the like, non-invasive diagnosis without radiation exposure becomes possible.

An organism has a characteristic of absorbing light in addition to a characteristic of scattering light. Therefore most of the light that enters an organism stops its straight propagation at about several mm depth, and is then scattered. In the case of an organism having a several cm thickness, for example, light is scattered considerably (multiple scattering) and propagates over a wide range in the organism.

Diagnosis using near infrared light is used for diagnosing breast cancer. Non-patent Literature 1 discloses an apparatus that allows near infrared light to enter a breast, detects the light that was scattered and propagated in the breast, and acquires a light absorption characteristic (absorption coefficient) and a light scattering characteristic (scattering coefficient) of an area where the light is scattered and propagated, and acquires functional information, such as oxygen saturation.

The apparatus according to Non-patent Literature 1 measures the optical characteristics and the functional information of a cancer site, which are different from those of a normal site, which suggests that cancer can be diagnosed by detecting the changes of the optical characteristics and functional information of the cancer site.

Patent Literature 1 discloses an apparatus that irradiates light onto an object, detects an acoustic wave generated from a cancer site which absorbed the propagated light, determines a sound pressure (initial sound pressure) at the moment when light was irradiated, and determines an absorption coefficient of the cancer from this sound pressure. This apparatus more accurately converts the initial sound pressure into the absorption coefficient by determining the average optical characteristic of the organism.
Patent Literature 1: Japanese Patent Application Laid-open No. 2010-88873
Non-Patent Literature 1: Natasha Shah et al., “The role of diffuse optical spectroscopy in the clinical management of breast cancer”, Disease Markers Vol. 19, (2003, 2004), No. 2-3, p. 95-105

### SUMMARY OF THE INVENTION

A breast has a chest wall near the base thereof. The chest wall includes muscles (e.g. pectoralis major muscle, intercostal muscles) having a high absorption coefficient. If an area near the chest wall is measured using the apparatus according to Non-patent Literature 1, part of the irradiated light scatters and reaches the chest wall, and a large amount of energy is absorbed. As a result, light after the large amount of energy is absorbed is detected, and the optical characteristic (absorption coefficient) to be calculated changes. In other words, the accuracy of the determined optical characteristics and functional information drops.

In the case of the apparatus according to Patent Literature 1 as well, accuracy to estimate the quantity of light drops due to the high absorption coefficient of the chest wall. This results in a drop in calculation accuracy of the optical characteristics and functional information.

As described above, the influence of the absorption of light energy by the chest wall on the measurement accuracy has been a problem in diagnosis using light. Therefore it has been demanded to enable good diagnosis by reducing this influence.

With the foregoing in view, it is an object of the present invention to reduce the influence of the chest wall on the measurement when the optical characteristics of the breast are acquired using light.

The present invention in its one aspect provides an object information acquiring apparatus, comprises an irradiation unit configured to irradiate light from a light source onto a breast, which is an object; a detection unit configured to detect a signal propagating from the breast by the light irradiation from the irradiation unit; a calculation unit configured to calculate an optical characteristic inside the breast based on the signal detected by the detection unit, wherein the irradiation unit configured to irradiate the light onto the breast in a direction away from a chest wall located at the base of the breast.

The present invention in its another aspect provides a control method for an object information acquiring apparatus having an irradiation unit, a detection unit and a calculation unit, the method comprises an irradiation step of operating the irradiation unit to irradiate light from a light source onto a breast, which is an object; a detection step of operating the detection unit to detect a signal propagating from the breast by the light irradiation from the irradiation unit; and a calculation step of operating the calculation unit to calculate an optical characteristic inside the breast based on the signal detected by the detection unit, wherein in the irradiation step, the irradiation unit irradiates light onto the breast in a direction away from the chest wall located at the base of the breast.

According to the present invention, the influence of the chest wall on the measurement can be reduced when the optical characteristics of the breast are acquired using light.

Further features of the present invention will become apparent from the following description of exemplary embodiments with reference to the attached drawings.

### BRIEF DESCRIPTION OF THE DRAWINGS

FIG. 1A is a diagram depicting an overview of an apparatus of Embodiment 1;

FIG. 1B is a top view depicting an overview of the apparatus of Embodiment 1;

FIG. 2 is a schematic diagram depicting a relationship of a chest wall and a holding unit;

FIG. 3A is an enlarged view of an example of an area near a light irradiation unit;

FIG. 3B is an enlarged view of another example of an area near the light irradiation unit;

FIG. 3C is an enlarged view of another example of an area near the light irradiation unit;

FIG. 4A is a diagram depicting an effect of the Embodiment 1;

FIG. 4B is another diagram depicting an effect of the Embodiment 1;

FIG. 4C is another diagram depicting an effect of the Embodiment 1;

FIG. 5A is a diagram depicting a system used for confirming the effect of Embodiment 1;

FIG. 5B is another diagram depicting a system used for confirming the effect of Embodiment 1;

FIG. 6A is a graph depicting the effect of Embodiment 1;

FIG. 6B is another graph depicting the effect of Embodiment 1;

FIG. 7 is a flow chart depicting a measurement which a control unit executes according to Embodiment 1;

FIG. 8 is a diagram depicting an overview of an apparatus according to Embodiment 2;

FIG. 9 is a flow chart depicting a measurement which a control unit executes according to Embodiment 2;

FIG. 10 is a diagram depicting an overview of an apparatus according to Embodiment 3; and

FIG. 11 is a flow chart depicting a measurement which a control unit executes according to Embodiment 3.

### DESCRIPTION OF THE EMBODIMENTS

Preferred embodiments of the present invention will be described with reference to the drawings. The dimensions, materials, shapes, relative arrangements or the like of the composing elements to be described hereinbelow should be changed appropriately depending on the configuration and the various conditions of the apparatus to which the invention is applied, and are not intended to limit the scope of the invention to the following description.

An object information acquiring apparatus of the present invention is an apparatus that utilizes the photoacoustic effect, where an acoustic wave, that is generated by irradiating the light (electromagnetic wave) onto an object and propagated in the object, is received, and object information, which is characteristic information on the object, is acquired as image data. This imaging technology is called “photoacoustic tomography” (PAT). The acquired object information is characteristic information to indicate the source distribution of an acoustic wave generated by light irradiation, the initial sound pressure distribution inside the object, the light energy absorption density distribution and the absorption coefficient information derived from the initial sound pressure distribution, and the concentration distribution of a substance which constitutes a tissue, for example. The substance which constitutes a tissue is, for example, the blood components indicated in the oxygen saturation distribution and in the oxyhemoglobin/deoxyhemoglobin concentration distribution, lipids, collagen or water.

The acoustic wave in the present invention is typically an ultrasound wave, including an elastic wave, such as a sound wave and an acoustic wave. An acoustic wave generated by the photoacoustic effect is called a “photoacoustic wave” or a “light-induced ultrasound wave”. In the apparatus of the present invention, an acoustic wave generated or reflected and propagated in the object is received by an acoustic wave detector, such as a probe.

The object information acquiring apparatus of the present invention is also an apparatus that detects light propagating inside an object after being irradiated onto the object, and determines the optical characteristic value distribution inside the object from the intensity thereof. The object information in this case is an average optical coefficient, absorption coefficient and scattering coefficient inside the object, and such functional information as oxygen saturation. The technology to acquire the optical characteristic values and generating image data inside the object from the optical characteristic values is called “diffuse optical tomography” (DOT).

In the following description, an object information acquiring apparatus using the principles of DOT will be described first as an application example of the present invention. Then an object information acquiring apparatus using the principles of PAT will be described as another application example. However the application targets of the present invention are not limited to these examples. For example, the present invention can be applied to an apparatus that acquires characteristic information for forming an image and storing this information in a memory. The present invention can also be understood as a control method of the object information acquiring apparatus and a program that allows an information processor to execute this control method, as described below.

### Embodiment 1

In Embodiment 1, an object information acquiring apparatus that acquires the average optical characteristic of an area where light is propagated, based on the principles of DOT, will be described. By applying the present invention to such an apparatus, the influence of the chest wall on measurement can be reduced, and optical characteristics can be acquired with high precision.

(Apparatus Configuration)

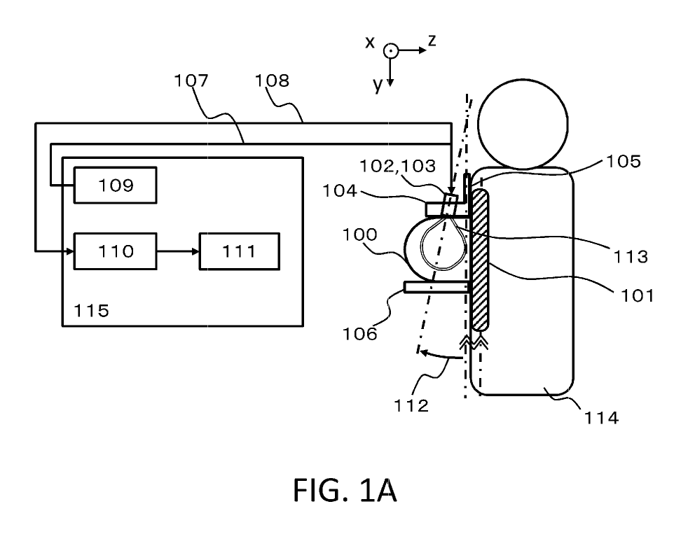

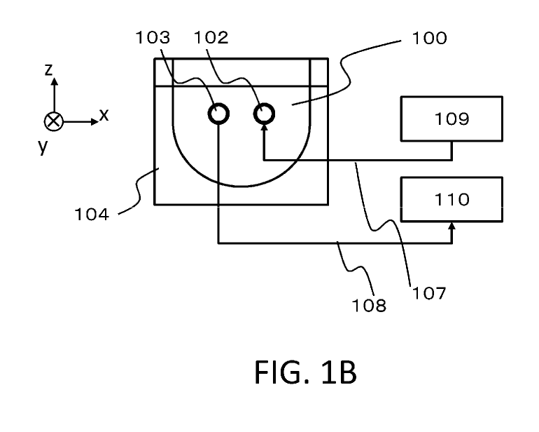

FIG. 1A shows an overview of the object information acquiring apparatus according to this embodiment. Each composing element will now be described.

A measurement object of the apparatus is a breast 100 of a testee 114. There is a chest wall 101 at the base of the breast 100, on the side of the body cavity of the testee 114.

The apparatus has a light irradiation unit 102, a light detection unit 103, a holding unit 104, a plane portion 105 (a part of the holding unit 104), a placement unit 106, an irradiation light guiding unit 107, a detected light guiding unit 108, a light source 109, a photodetector 110, an optical coefficient calculation unit 111, and a control unit 115.

The light irradiation unit 102 irradiates light onto a breast 100. For the light irradiation unit 102, an optical fiber end or an optical element, such as a collimator or focuser, can be used. The light irradiation unit 102 is disposed such that an angle of the irradiation light, from a substantially flat plane extending from the chest wall 101 (irradiation angle), can be set to the angle 112 using the holding unit 104. Thereby the light can be irradiated in a direction away from the substantially flat plane extending from the chest wall 101. In this embodiment, the light irradiation unit 102 corresponds to the irradiation unit of the present invention.

The irradiation light guiding unit 107 guides light from the light source 109 to the light irradiation unit 102. An optical fiber or an optical element can be used for the irradiation light guiding unit 107.

The light detection unit 103 detects light that reached the light detection unit after being irradiated from the light irradiation unit 102 onto the breast 100, and propagating inside the breast. For the light detection unit 103, an optical fiber end or an optical element, such as a collimator or a focuser, can be used. In this embodiment, the light detection unit 103 corresponds to the detection unit of the present invention.

In FIG. 1A, the light detection unit 103, which is located behind the light irradiation unit 102, is not indicated. Therefore the light detection unit 103 is illustrated in FIG. 1B. FIG. 1B is a diagram when FIG. 1A is viewed from the top (the head side of the testee 114). The coordinate system and the reference numerals of FIG. 1B are the same as FIG. 1A.

The detected light guiding unit 108 guides the light detected by the detection unit 103 to the photodetector 110. An optical fiber or an optical element can be used for the detected light guiding unit 108.

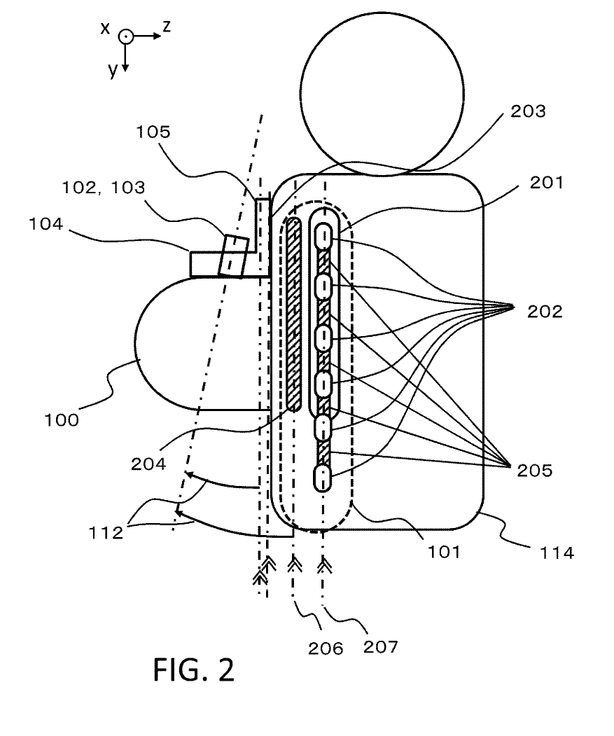

In the holding unit 104, the light irradiation unit 102 is disposed such that the light is irradiated at the angle 112 from the chest wall 101. The holding unit 104 has a plane portion 105 which is a plane segment. The plane portion 105 will be described with reference to FIG. 2. The plane portion 105 is along the body surface 203 of the testee 114, above the body of sternum 201 and the costal cartilages 202 constituting the chest wall 101. The substantially flat plane 206 which extends the muscles that constitute the chest wall 101 (e.g. pectoralis major muscle 204, intercostal muscles 205) and the substantially flat plane 207 formed by the body of sternum 201 and the costal cartilages 202 are approximately parallel. The body surface 203 is supported by the body of sternum 201 and the costal cartilages 202 directly under the body surface 203, so the body surface 203 is approximately parallel with the substantially flat plane 207 formed by the body of sternum 201 and the costal cartilages 202. Therefore if the plane portion 105 is along the body surface 203 directly above the body of sternum 201 and the costal cartilages 202, the angle 112 is formed between the light irradiation unit 102 and the muscles (205, 206), which are the main light absorber of the chest wall 101.

In FIG. 2, the composing elements denoted with the reference numerals 201 to 206 extend in the x axis direction (direction perpendicular to the paper surface) indicated in FIG. 2. In other words, the extending direction of the substantial plane is approximately parallel with the xy plane. The reference numerals 206 and 207 indicate a cross-section of the extending plane. In this embodiment, the holding unit 104 corresponds to the holding unit of the present invention.

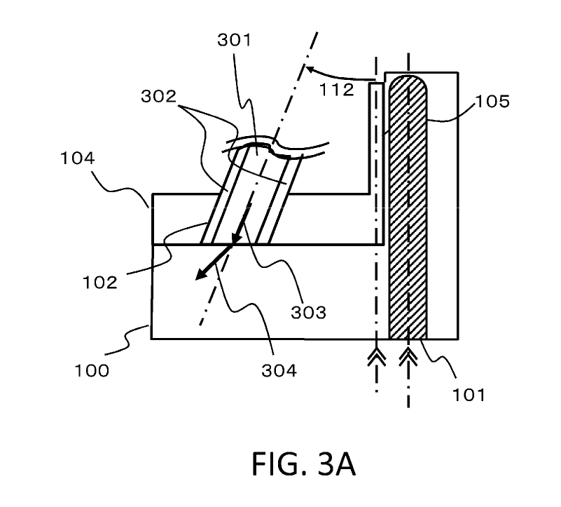

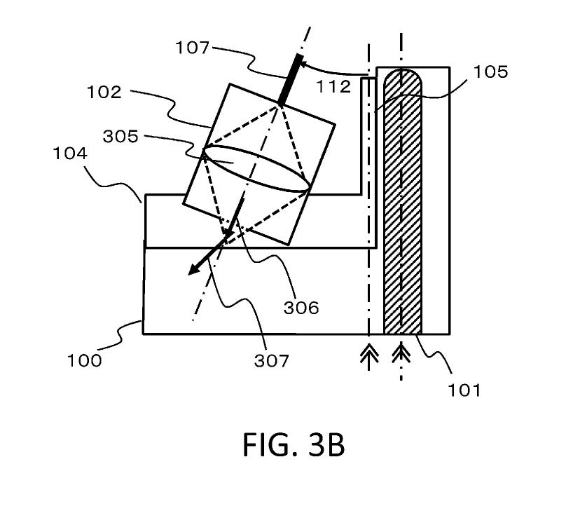

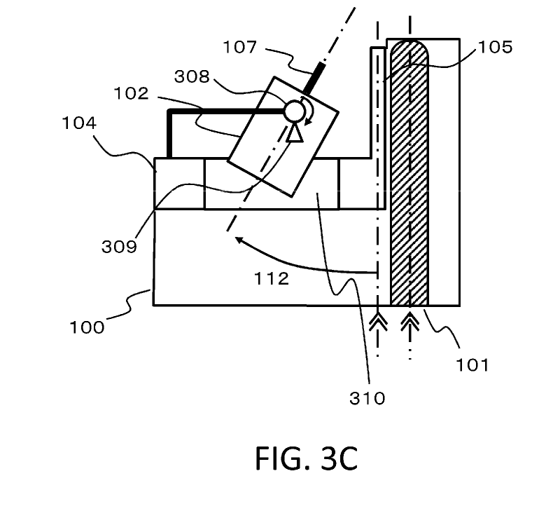

The configuration of the light irradiation unit 102 and the holding unit 104 will now be described with reference to FIG. 3A to FIG. 3C, which are enlarged views of an area near the light irradiation unit 102. In FIG. 3A to FIG. 3C, a composing element the same as that of FIG. 1A is denoted with the same reference numeral.

FIG. 3A shows a case when the light irradiation unit 102 is an optical fiber end. The end of the light irradiation unit 102 is parallel with a plane where the holding unit 104 contacts the breast 100. The optical fiber includes a core 301 and a cladding 302. The light irradiation unit 102 is set such that the primary axis of the optical fiber end forms an angle 112. The arrow 303 indicates an incident direction of light that enters the breast 100 in the primary axis direction of the optical fiber, and the arrow 304 indicates a direction in which the light refracts from the incident direction 303 after entering the breast. The material of the core 301 is quartz, glass, plastic or the like, and the refractive index of the core 301 is higher than that of the breast 100 since the breast 100 is an organism containing a lot of water. Typically, the refractive index of the core 301 is approximately 1.45 and the refractive index of the breast 100 is approximately 1.33. This means that the refraction direction 304 has an angle greater than the angle 112. In other words, the light enters in a direction away from the chest wall 101.

FIG. 3B shows a case when the light irradiation unit 102 is a focuser. A lens 305 collects the light of the irradiation light guiding unit 107 to a surface where the holding unit 104 and the breast 100 contact. Therefore it is preferable that the holding unit 104 is formed from a material whose light transmittance is high. Acrylic, for example, can be used. The arrow 306 indicates an incident direction 306 of light that enters the breast 100 in the optical axis direction of the lens 305, and the arrow 307 indicates a direction in which the incident direction is refracted. If the material of the holding unit 104 is acrylic, the refractive index of acrylic is approximately 1.49, and the refractive index of the breast 100 is approximately 1.33, which means that the refraction direction 307 has an angle greater than the angle 112. In other words, the light enters in a direction away from the chest wall 101.

The light irradiation unit 102 may be disposed to be movable with respect to the holding unit 104. FIG. 3C is an example when the light irradiation unit 102 is movable. The light irradiation unit 102 can be rotated by a rotation unit 308 which is fixed in the holding unit 104. A fixing unit 309 can change the irradiation angle by switching the rotation state of the rotation unit 308. The fixing unit 309 is fixed after rotating the light irradiation unit 102 to an angle to obtain a desired angle 112. Considering a movable range of the light irradiation unit 102, it is desirable that there is an opening 310 in a part of the holding unit 104. If the light irradiation unit 102 is movable, the angle 112 can be changed as necessary, for example. A case of changing the angle 112 is, for example, when it is known that the chest wall 101 is in a position closer to the breast by ultrasound inspection or MRI inspection in advance, and the irradiation light must be further away from the chest wall. In this case, the irradiation angle to be set is increased.

The light which entered like this becomes the scattered light 113 in FIG. 1A, which propagates inside the breast 100.

The placement unit 106 supports the breast 100. The breast 100 is placed on the placement unit 106 such that the breast 100 contacts the holding unit 104. The configuration of the apparatus, however, is not limited to a mode of the testee 114 standing up straight and placing their breast 100 on the placement unit 106. For example, the present invention can also be applied to a mode where the testee 114 is lying face down on a bed, and measuring the breast 100 that is hanging downward. Even in the case of the testee 114 lying face up on a bed for measurement, the present invention can be applied by holding the breast in an appropriate shape using the placement unit 106 and the holding unit 104.

The light source 109 generates light that is irradiated from the light irradiation unit 102. The light source 109 generates at least one of CW light, pulsed light and intensity-modulated light. The pulse width of the pulsed light is preferably of a pico second order. The modulation frequency of the intensity-modulated light is preferably of a several tens kilohertz to several gigahertz order. For a light emitting source that generates light, a halogen lamp that includes light in a near infrared region or a laser and a light emitting diode, of which wavelength is in the near infrared region, can be used. For the laser, various lasers can be used, such as a solid-state laser, a gas laser, a dye laser and a semiconductor laser. To generate a pulsed light, the light source 109 includes a short pulse driving device or circuit, whereby the light emitting source is driven by short pulses. To generate the intensity-modulated light, the light source 109 includes an intensity-modulation driving device or circuit, whereby the light emitting source is driven by intensity modulation. In this embodiment, the light of the light source 109 is guided to the light irradiation unit 102 using the irradiation light guiding unit 107. But, in a case where the light source 109 is small (e.g. semiconductor laser), the light source 109 may be installed inside the light irradiation unit 102.

The photodetector 110 converts the light detected by the light detection unit 103 into a signal, such as an electric signal. For a photodetection element included in the photodetector 110, a photomultiplier tube (PMT), an avalanche photodiode (APD), a photodiode (PD) or the like can be used.

If the light source 109 generates pulsed light, it is preferable that the photodetector 110 be able to carry out photon counting. By photon counting, the SN can be improved even if the intensity of the light has become weak due to scattering and absorption while propagating in the breast 100. To carry out photon counting, the photodetector 110 includes a device or circuit that is required for photon counting (e.g. wave height discriminator, pulse count circuit, photodetection element cooler). If the light source 109 generates pulsed light, the photodetector 110 detects light in a time series, and outputs a time profile (Time-Of-Flight: TOF) of the light intensity as a signal.

If the light source 109 generates intensity-modulated light, it is preferable that the photodetector 110 can carry out wave detection. Homodyne detection or heterodyne detection can be used for the wave detection. By wave detection, even the intensity-modulated light, of which modulated amplitude has become very weak while the light propagates in the breast 100, can be measured with high SN. In the case of performing wave detection, the photodetector 110 includes a device or circuit required for the wave detection (e.g. lock-in amplifier or spectrum analyzer). If the light source 109 generates intensity-modulated light, the photodetector 110 outputs the amplitude and the phase of the intensity modulation as a signal. In this embodiment, the light is guided from the light detection unit 103 to the photodetector 110 using the detected light guiding unit 108, but if the photodetector 110 is small, the photodetector 110 may be installed inside the light detection unit 103 without installing the detected light guiding unit 108. Only the photodetection element included in the photodetector 110 may be disposed in the light detection unit 103.

The optical coefficient calculation unit 111 calculates an average optical characteristic of an area inside the breast 100 where the light propagated, using a signal from the photodetector 110. The optical coefficient calculation unit 111 can calculate at least one of an absorption coefficient μa, a scattering coefficient μs′, and an effective attenuation coefficient μeff using a light diffusion equation that describes the behavior of light in a medium, such as an organism, that scatters and absorbs light.

If the photodetector 110 outputs TOF, the absorption coefficient μa and the scattering coefficient μs′ are calculated using the light diffusion equation in a time domain. For example, the breast 100 held between the holding unit 104 and the placement unit 106 is regarded as having a slab shape, a slab shape analytical solution of the light diffusion equation in the time domain is fitted to TOF, and μa and μs′ at optimum fitting are regarded as the measured values.

If the photodetector 110 outputs the intensity-modulated amplitude and phase, then the calculated amplitude and the calculated phase obtained from the slab shape analytical solution of the light diffusion equation in the frequency domain are fitted to the detected amplitude and the phase, and μa and μs′ at the optimum fitting are regarded as the measured values. If the light source 109 generates CW light and the photodetector 110 outputs the intensity of the CW light, μeff can be obtained by the method disclosed in Patent Literature 1.

The optical coefficient calculation unit 111 may be a program installed on a computer or may be an electronic circuit.

The control unit 115 controls the measurement of the breast 100. For the control unit 115, a PC or an electric circuit can be used. The light source 109, the photodetector 110 and the optical coefficient calculation unit 111 may be connected with the control unit 115 by wire or may be enclosed in the control unit 115.

(Optical Characteristic Acquisition)

Next reducing the influence of the chest wall on the measurement and acquiring the optical characteristics with high precision by the above mentioned configuration is described.

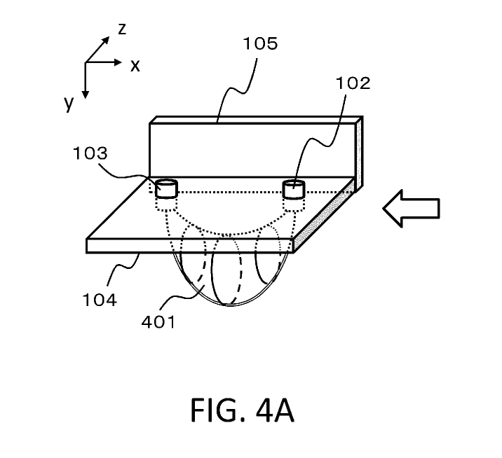

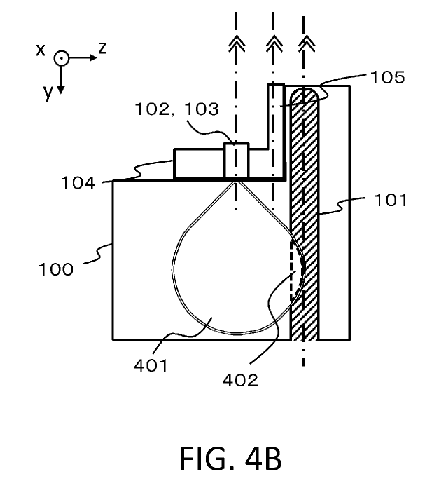

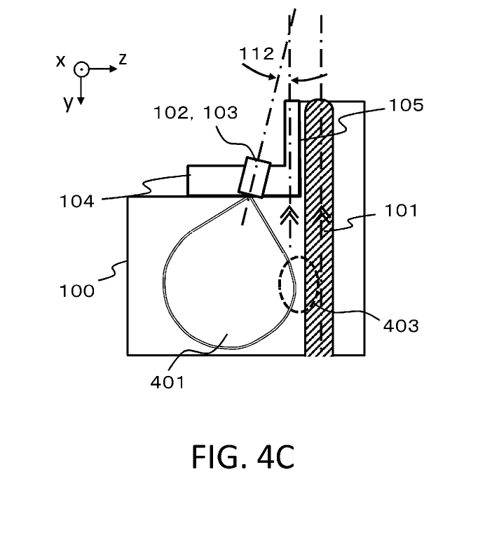

FIG. 4A to FIG. 4C are diagrams viewing an area around the holding unit 104 in the same coordinate system as FIG. 1A and FIG. 1B. A composing element the same as that in FIG. 1A and FIG. 1B is denoted with the same reference numeral. It is known that an envelope curve of a path of each light (photon) irradiated from the light irradiation unit 102 and detected by the light detection unit 103 becomes a curved spindle area that is included in the double lines 401. The optical coefficient calculation unit 111 calculates an average optical coefficient inside the curved spindle area 401.

FIG. 4B is a diagram viewed from the arrow direction shown in FIG. 4A, when the light irradiation unit 102 is disposed parallel with the plane portion 105. In FIG. 4B, a part 402 of the curved spindle area overlaps with the chest wall 101 (overlapping area 402). Since the chest wall is constituted primarily by muscles whose absorption coefficient is high, a lot of energy of the light is absorbed in the overlapping area 402. Therefore the optical coefficient calculation unit 111, which calculates the average optical coefficient in the curved spindle area 401, calculates the absorption coefficient as if the absorption coefficient is high in the entire curved spindle area 401.

FIG. 4C is a diagram viewed from the arrow direction shown in FIG. 4A, when the light irradiation unit 102 is inclined at the angle 112. In FIG. 4C, the light irradiation unit 102 is disposed inclined at the angle 112, whereby the entire curved spindle area 401 is moved in a direction away from the chest wall 101. Therefore as indicated by the reference numeral 403, the curved spindle area 401 is less likely to overlap with the chest wall 101. As a result, the optical coefficient calculation unit 111 calculates the average optical coefficient of the curved spindle area 401 which is less likely to overlap with the chest wall 101. In other words, it can be prevented that the average optical coefficient is calculated to be higher than the actual value due to the influence of the chest wall 101, and therefore the average optical coefficient can be calculated with high precision.

As FIG. 4C shows, if the light irradiation unit 102 is inclined at the angle 112, the curved spindle area 401 is shifted to the angle 112 direction. This means that the light detection efficiency can be improved if the light detection unit 103 is inclined at the angle 112, hence it is preferable that the light detection unit 103 is inclined at the angle 112.

(Effect)

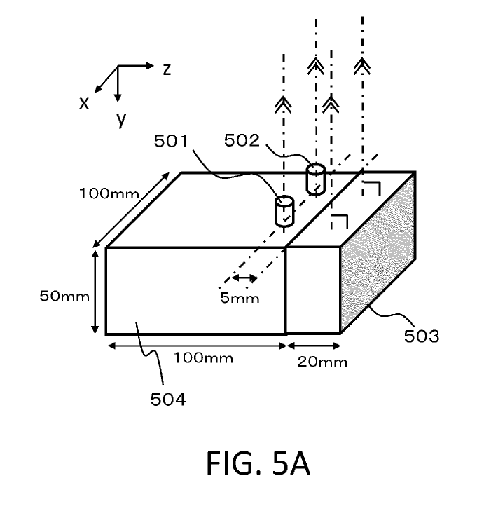

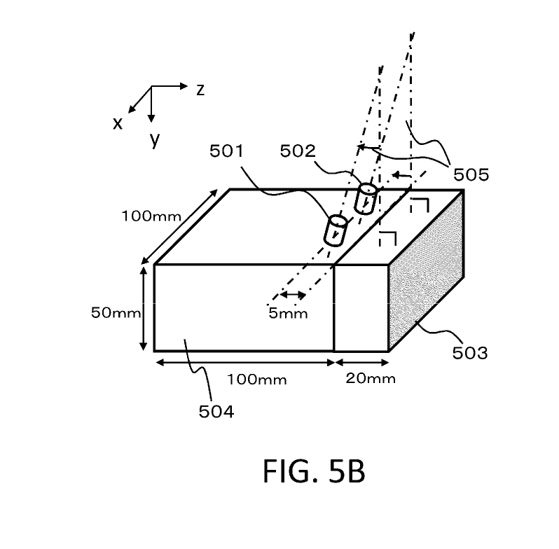

The effect of Embodiment 1 will be described using theoretical calculations. FIG. 5A and FIG. 5B show a calculation system. A configuration common to FIG. 5A and FIG. 5B will be described. The reference numeral 501 indicates the light irradiation unit which corresponds to the reference numeral 102 in FIG. 1A. The reference numeral 502 indicates the light detection unit which corresponds to the reference numeral 103 in FIG. 1A. The reference numeral 503 indicates the chest wall which corresponds to the reference numeral 101 in FIG. 1A. And the reference numeral 504 indicates the breast which corresponds to the reference numeral 100 in FIG. 1A.

The optical coefficients (true values) of the breast 504 are absorption coefficient μa=0.0055 [/mm] and scattering coefficient μs′=0.96 [/mm]. The optical coefficients (true values) of the chest wall 503 are absorption coefficient μa=1.1 [/mm] and scattering coefficient μs′=0.96 [/mm]. For these optical coefficients, numerical values appropriate for an organism are used. FIG. 5A is a case when no angle is formed between the light irradiation unit 501 and the light detection unit 502, which corresponds to FIG. 4B. FIG. 5B is a case when the light irradiation unit 501 and the light detection unit 502 form an angle 505, which corresponds to FIG. 4C. For FIG. 5B, both cases when the angle 505 is 20° and 30° are calculated.

A method for calculating the optical coefficients will be described. In the first calculation step, the TOF is calculated by the Monte Carlo method using the system in FIGS. 5A and 5B. The Monte Carlo method is a method for tracking a propagation optical path of each photon by causing scattering and absorption. According to this method, the behavior of a photon, reflecting the angle of the light irradiation unit 501 and the difference of optical coefficients between the breast 504 and the chest wall 503, can be calculated.

In the second calculation step, the TOF acquired by the first Monte Carlo method is fitted to the slab shape analytical solution of the light diffusion equation. Variables that are changed in the fitting are the absorption coefficient μa and the scattering coefficient μs′. The absorption coefficient μa and the scattering coefficient μs′ when the optimum fitting is determined are regarded as the calculated values of the optical coefficients. The second calculation step corresponds to the calculation by the optical coefficient calculation unit 111. In this embodiment, the optical coefficient calculation unit 111 corresponds to the calculation unit of the present invention.

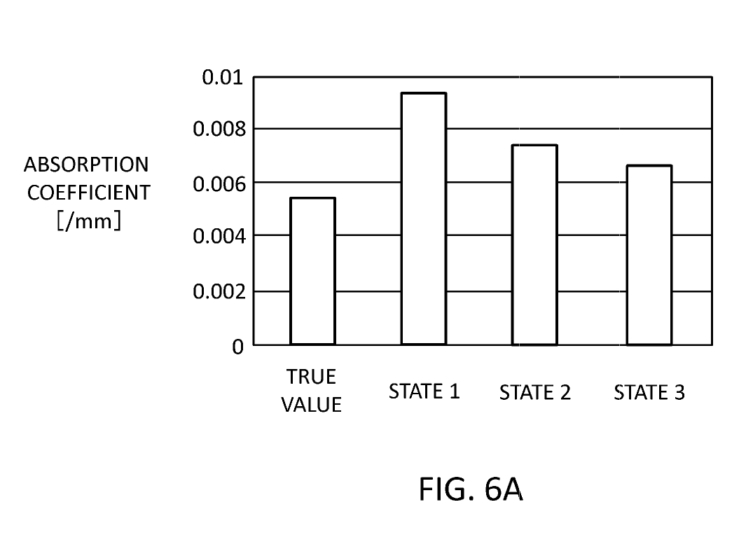

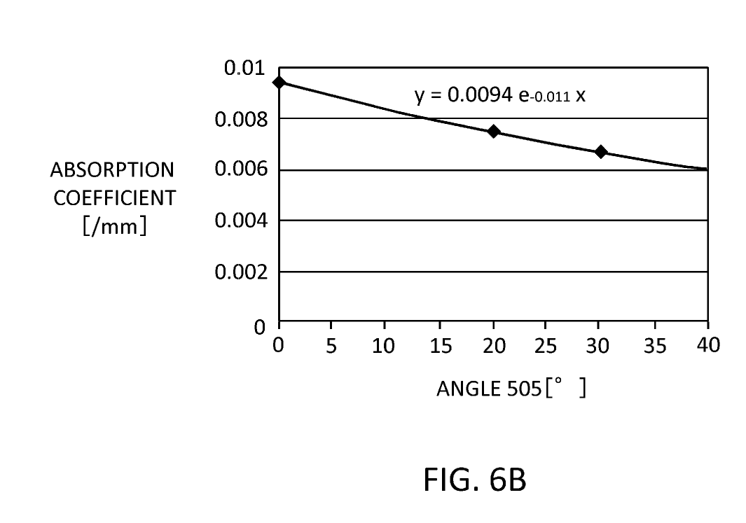

FIG. 6A is a graph comparing the absorption coefficient μa between the true value and the calculated values in the case of FIG. 5A (state 1), in the case of FIG. 5B where the angle 505 is 20° (state 2), and in the case of FIG. 5B where the angle 505 is 30° (state 3). In both cases when the angle 505 is 20° and 30°, the absorption coefficients become closer to the true value since the influence of the chest wall on the measurement values is reduced. If the angle 505 is not formed, the calculation error rate of the absorption coefficient μa is +71%. On the other hand, forming a 20° to 30° angle 505 improves the calculation error rate to a +22% to +36% range.

FIG. 6B is a graph when the angle 505 is plotted on the abscissa, and the calculated value of the absorption coefficient μa is plotted on the ordinate, where a dot indicates the calculated value and a curve indicates an approximate curve. As the approximate curve indicates, the angle 505 when the error rate with respect to the true value of the absorption coefficient is +30% is 24.2°, and the angle 505 when the error rate is +10% is 39.0°. Therefore to obtain the absorption coefficient μa at a 30% or less error, the angle 505 is preferably 24.2° or more. It is even more preferable that the angle 505 is 39.0° or more, then the absorption coefficient μa can be acquired at a 10% or less error rate.

(Measurement Flow)

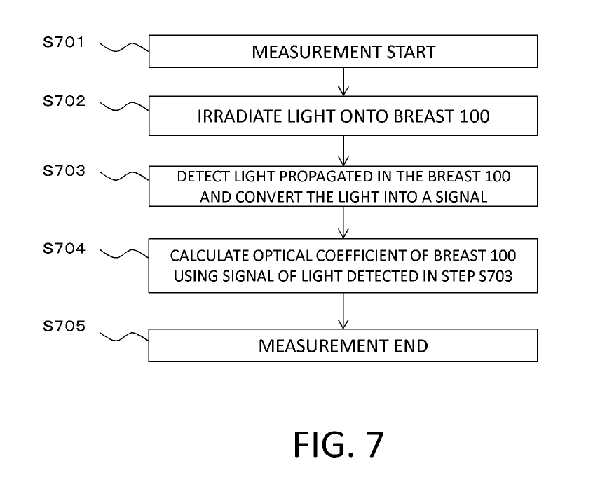

The measurement flow executed by the control unit 115 in Embodiment 1 will be described with reference to FIG. 7.

In step S701, the measurement starts.

In step S702, the light generated in the light source 109 is guided to the light irradiation unit 102 by the irradiation light guiding unit 107, and is irradiated onto the breast 100 held by the holding unit 104 and the placement unit 106.

In step S703, the light irradiated onto the breast 100 in S702 and propagated inside the breast 100 is detected by the light detection unit 103. The light detected by the light detection unit 103 is guided to the photodetector 110 by the detected light guiding unit 108. The photodetector 110 converts the detected light into a signal. To improve the SN of the signal, signals may be integrated by repeating step S702 and step S703.

In step S704, the average optical coefficient of the breast 100 is calculated by the optical coefficient calculation unit 111 using the signal obtained in step S703.

In step S705, the measurement ends.

As described above, according to Embodiment 1, an average optical coefficient of the breast can be acquired with high precision by reducing the influence of the chest wall on the measurement values.

### Embodiment 2

In Embodiment 2, an object information acquiring apparatus that detects an acoustic wave generated from a light absorber (e.g. cancer) in a breast by irradiating light onto the breast, and determining a sound pressure distribution inside the breast at the moment when light was irradiated (initial sound pressure distribution) based on the principles of PAT, will be described. The object information acquiring apparatus of this embodiment can also be called a “photoacoustic imaging apparatus” that determines an absorption coefficient distribution, which is an optical characteristic distribution inside the breast, from the initial sound pressure distribution. In the following description, an apparatus that can acquire the absorption coefficient distribution with high precision by reducing the influence of the chest wall on the measurement in particular will be described.

(Apparatus Configuration)

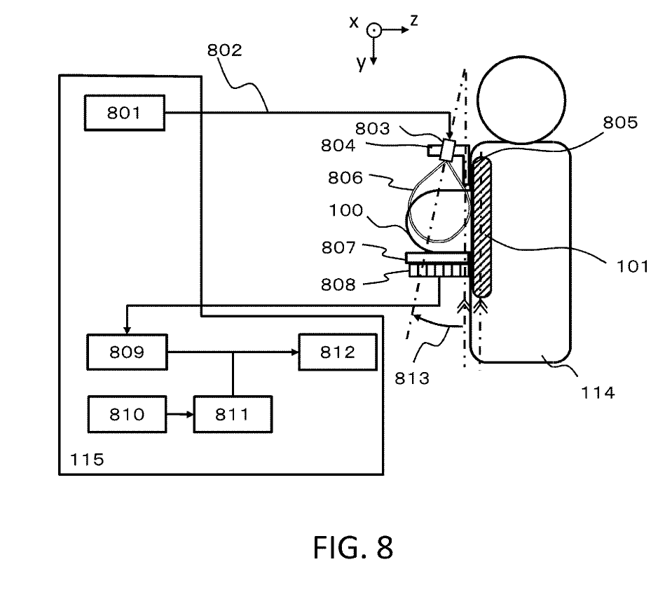

FIG. 8 shows an overview of the apparatus according to Embodiment 2 of the present invention. A configuration to acquire image information of an object will be described below. In FIG. 8, a composing element the same as that of Embodiment 1 is denoted with the same reference numeral as FIG. 1, for which description is omitted.

The apparatus has a light source 801, an irradiation light guiding unit 802, a light irradiation unit 803, a holding unit 804 (including a plane portion 805), a placement unit 807, and a transducer array 808. For the control unit 115, the apparatus also has a reconstruction unit 809, an optical coefficient setting unit 810, a light intensity distribution calculation unit 811, and an absorption coefficient distribution calculation unit 812.

The light source 801 generates light which the light irradiation unit 803 irradiates onto the breast 100. The light source 801 includes at least one coherent or incoherent pulsed light source. To generate a photoacoustic effect, the pulse width is preferably several hundred nanoseconds or less. For the light source, a laser which can emit high power is preferable, but a light emitting diode or the like may be used instead of a laser. For the laser, various lasers including a solid-state laser, a gas laser, a dye laser and a semiconductor laser can be used.

The irradiated light guiding unit 802 guides light from the light source 801 to the light irradiation unit 803. For the irradiation light guiding unit 802, an optical fiber or an optical element can be used.

The light irradiation unit 803 irradiates light from the light source 801 onto the breast 100 by a method appropriate for the photoacoustic measurement. To increase the SN ratio of the received signal, the light may be irradiated not only onto one surface of the object but onto a plurality of surfaces. For example, an irradiation port may also be created on the side where a transducer array 808 exists. For the light irradiation unit 803, a mirror, a lens that collects, expands or changes the shape of the light, a prism that disperses, refracts and reflects the light, an optical fiber end or the like can be used. The light irradiation unit 803 is disposed so as to be inclined from the holding unit 804 at the angle 813. Thereby the light can be irradiated in a direction away from a substantial plane which extends from the chest wall 101. In this embodiment, the light irradiation unit 803 corresponds to the irradiation unit of the present invention.

In this embodiment, the light of the light source 801 is guided to the light irradiation unit 803 using the irradiation light guiding unit 802. However if the light source 801 is small (e.g. a semiconductor laser), the light source 801 may be installed inside the light irradiation unit 803 without installing the irradiation light guiding unit 802.

The holding unit 804 specifies an angle of the light irradiated from the light irradiation unit 803 to the angle 813 away from the chest wall 101. If the irradiated light spreads to a certain degree, the angle 813 is specified using the optical axis at the center. The holding unit 804 has a plane portion 805, which is a plane segment. The function of the plane portion 805 is the same as the plane portion 105 of Embodiment 1. If the light irradiation unit 803 is inclined at the angle 813 using the holding unit 804, the area 806 inside the breast 100 where light propagates is shifted away from the chest wall 101. This is identical to the feature described with respect to the holding unit 104 in Embodiment 1. A configuration that allows changing the angle 813 may be used, just like Embodiment 1. Then the light irradiation angle in accordance with the size and holding state of the breast can be set. In this embodiment, the holding unit 804 corresponds to the holding unit of the present invention.

The placement unit 807 supports the breast 100. The transducer array 808 receives an elastic wave via the placement unit 807, hence it is preferable that the material of the placement unit 807 has an acoustic characteristic matching with the breast 100 and the transducer array 808. It is also preferable to use an acoustic matching material.

The transducer array 808 receives an elastic wave generated inside the breast 100 onto which the light was irradiated, and converts the elastic wave into a signal, such as an electric signal. For the transducer array 808, a transducer using piezoelectric phenomena, a transducer using the resonance of light, a transducer using a change of capacity or the like can be used. Any transducer can be used if an elastic wave can be received and converted into a signal. The transducer array 808 receives an elastic wave at different positions, hence it is preferable to include a plurality of transducers (elements). In this embodiment, the transducer array 808 corresponds to the detection unit of the present invention.

The reconstruction unit 809 reconstructs the initial sound pressure distribution in the breast 100 at the moment when light was irradiated, using a plurality of signals outputted from the transducer array 808. The reconstruction unit 809 reconstructs the initial sound pressure distribution using, for example, a reverse projection method in a time domain or Fourier domain, which is commonly used in tomography technology.

The optical coefficient setting unit 810 sets the average optical coefficients (average absorption coefficient μa_a and average scattering coefficient μs′_a) of the breast 100 in the light intensity distribution calculation unit 811. For this setting, known statistical values of the optical coefficients of the breast corresponding to the age of the testee 114 can be used. For the optical coefficient values, the input values inputted externally by an operator, the values acquired from an internal memory, the measured values by DOT or the like may also be used.

The light intensity distribution calculation unit 811 calculates the light intensity distribution inside the breast 100 using the average absorption coefficient μa_a and average scattering coefficient μs′_a, which are set by the optical coefficient setting unit 810. For the calculation of the light intensity distribution, a numerical solution of a transport equation, a numerical solution of a diffusion approximation equation, a numerical solution by the Monte Carlo method or the like can be used.

The absorption coefficient distribution calculation unit 812 calculates the absorption coefficient distribution inside the breast 100 by correcting the initial sound pressure distribution by the light intensity distribution. In this embodiment, the reconstruction unit 809, the light intensity distribution calculation unit 811 and the absorption coefficient distribution calculation unit 812 correspond to the calculation unit of the present invention. In this embodiment, the optical coefficient setting unit 810 corresponds to the setting unit of the present invention.

Here the initial sound pressure P (r) at a position r is given by the following Expression (1).

P(r)=Γ·μa(r)·φ(r) Expression (1)

Γ is a Grueneisen coefficient determined by dividing a product of a volume expansion coefficient (β) and a square of the sound velocity (c) by a specific heat at constant pressure (Cp). μa (r) is an absorption coefficient at the position r. φ (r) is the light intensity at the position r. It is known that Γ becomes approximately a constant value according to the tissue of the organism.

The absorption coefficient distribution calculation unit 812 divides the initial sound pressure P (r) acquired by the reconstruction unit 809 by the light intensity φ (r) acquired by the light intensity distribution calculation unit 811 and a known Γ (constant value). Thereby the influence of attenuation of the light or the like can be corrected. As a result, the absorption coefficient μa (r) can be calculated. By performing the correction at each position, the absorption coefficient distribution inside the breast 100 is calculated.

The reconstruction unit 809, the optical coefficient setting unit 810, the light intensity distribution calculation unit 811 and the absorption coefficient distribution calculation unit 812 may be programs installed on a computer, or may be electronic circuits.

(Effect)

The effect of Embodiment 2 will be described. A case when a part of an area 806, where the light is propagating, overlaps with the chest wall 101 is considered.

The chest wall 101 having a high absorption coefficient absorbs lots of energy from the light (a photon) that reaches the chest wall 101. A part of a photon of which energy is absorbed is scattered inside the chest wall 101, propagates in the breast 100, and returns. Therefore compared with a case when the chest wall 101 does not exist, a photon of which energy is lower does exist in the breast 100 (particularly on the chest wall 101 side). This means that the light intensity drops in an area of the breast 100 near the chest wall 101, because of the presence of the chest wall 101. If this state is applied to Expression (1), the initial sound pressure P (r) drops since the light intensity φ (r) drops.

The light intensity distribution calculation unit 811, on the other hand, calculates the light intensity in the breast 100 using the average optical coefficients (μa_a, μs′_a), on which the influence of the chest wall is small or zero, set by the optical coefficient setting unit 810. Therefore it is difficult for the light intensity distribution calculation unit 811 to reflect the influence of the chest wall 101, which is spatially heterogeneous, on the light intensity distribution. As a result, the calculated value of the light intensity in an area near the chest wall 101 is greater than an actual value. The absorption coefficient distribution calculation unit 812 divides the initial sound pressure P (r), which dropped due to the influence of the chest wall, by φ (r), which is greater than the actual light intensity, hence the calculated value of the absorption coefficient μa (r) is smaller than the actual value.

However according to the present invention, the holding unit 804 specifies the angle of the position of the light irradiation unit 803 to an angle 813 away from the chest wall, therefore an overlapping area of the area 806 and the chest wall 101 is smaller. Hence the drop in the initial sound pressure P (r) due to the influence of the chest wall 101 can be reduced, and the calculation accuracy of the absorption coefficient μa (r) of the absorption coefficient distribution calculation unit 812 can be improved.

In the calculation of the absorption coefficient μa (r) of the system in FIG. 5 according to Embodiment 2, the calculation accuracy of the absorption coefficient μa (r) inside the breast 504 can be improved.

(Measurement Flow)

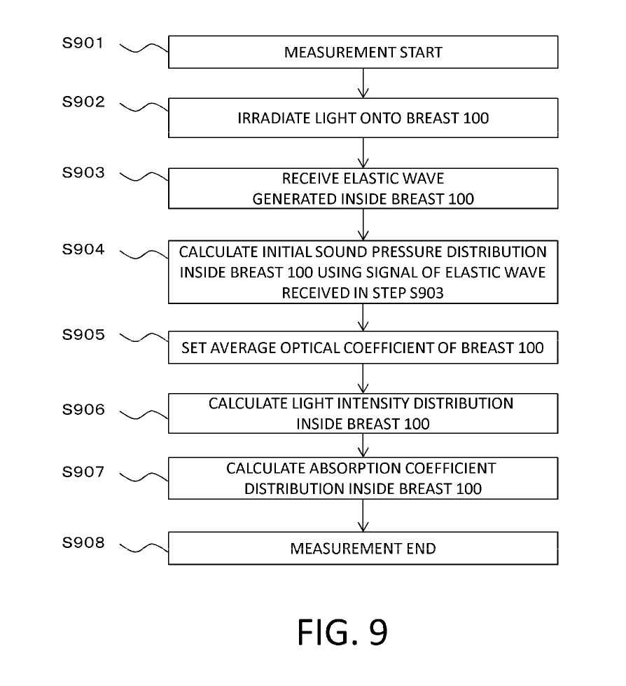

The measurement flow executed by the control unit 115 in Embodiment 2 will be described with reference to FIG. 9.

In step S901, the measurement starts.

In step S902, the light generated in the light source 801 is guided to the light irradiation unit 803 by the irradiation light guiding unit 802, and is irradiated onto the breast 100 held by the placement unit 807.

In step S903, the transducer array 808 receives an elastic wave generated by the light which was irradiated onto the breast 100 in step S902, and is absorbed by a light absorber (e.g. cancer) inside the breast, and converts the elastic wave into a signal. To improve the SN of the signal, the signals may be integrated by repeating step S902 and step S903.

In step S904, the reconstruction unit 809 calculates the initial sound pressure distribution inside the breast 100 using the signal of the elastic wave received in step S903.

In step S905, the optical coefficient setting unit 810 sets the average optical coefficients of the breast 100.

In step S906, the light intensity distribution calculation unit 811 calculates the light intensity distribution inside the breast 100 using the average optical coefficients which are set in step S905.

In step S907, the absorption coefficient distribution calculation unit 812 calculates the absorption coefficient distribution inside the breast 100 using the initial sound pressure distribution calculated in step S904 and the light intensity distribution calculated in step S906.

In step S908, the measurement ends.

As described above, according to Embodiment 2, the absorption coefficient distribution inside the breast 100 can be calculated with high precision by reducing the influence of the chest wall on the measurement values, and the absorption coefficient of the light absorber (e.g. cancer) inside the breast can be imaged with high precision.

### Embodiment 3

In Embodiment 3, an apparatus that irradiates light onto a breast detects an acoustic wave generated by the photoacoustic effect and determines the optical characteristic distribution inside the breast will be described. In this embodiment in particular, an average optical coefficient of the breast is determined by the light irradiation, and an absorption coefficient distribution, which is an optical characteristic distribution, is calculated using the average optical coefficient.

(Apparatus Configuration)

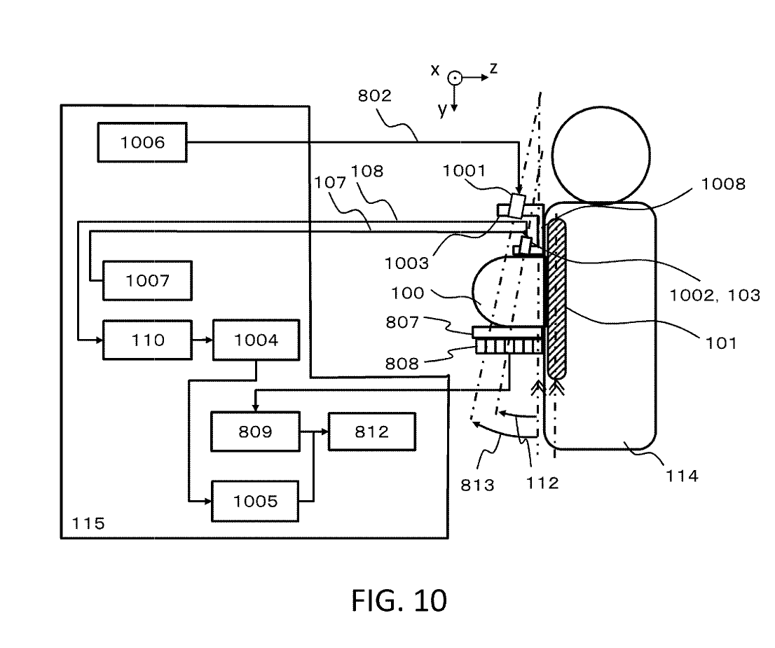

FIG. 10 shows an overview of the object information acquiring apparatus according to Embodiment 3. In FIG. 10, a composing element the same as that of Embodiment 1 or Embodiment 2 is denoted with the same reference numeral as FIG. 1 or FIG. 8, for which description is omitted.

As composing elements that are different from FIG. 1 or FIG. 8, the apparatus has a first light irradiation unit 1001, a second light irradiation unit 1002, a holding unit 1003 (including a plane portion 1008), an optical coefficient calculation unit 1004, a light intensity distribution calculation unit 1005, a first light source 1006 and a second light source 1007.

The first light irradiation unit 1001 has a same function as the light irradiation unit 803 in Embodiment 2.

The first light source 1006 has a same function as the light source 801 in Embodiment 2. And the first light source 1006 supplies light to the first light irradiation unit 1001. As a result, a photoacoustic effect is induced in the object and a photoacoustic wave is generated.

The second light irradiation unit 1002 has a same function as the light irradiation unit 102 in Embodiment 1. Just like Embodiment 1, the light detection unit 103, which is located behind the first light irradiation unit 102, is not indicated in FIG. 10. In this embodiment, the second light irradiation unit 1002 corresponds to the second irradiation unit of the present invention. In this embodiment, the light detection unit 103 corresponds to the second detection unit of the present invention.

The second light source 1007 has a same function as the light source 109 in Embodiment 1. And the second light source 1007 supplies light to the second light irradiation unit 1002. As a result, the light is irradiated for acquiring the optical characteristic. In this embodiment, the second light source 1007 corresponds to the second light source of the present invention.

The holding unit 1003 specifies the angle of the position of the second light irradiation unit 1002 to an angle 112 away from the chest wall 101. The holding unit 1003 also specifies the angle of the position of the first light irradiation unit 1001 to an angle 813 away from the chest wall 101. The holding unit 1003 has a plane portion 1008 which is a plane segment. The function of the plane portion 1008 is the same as the plane portion 105 in Embodiment 1. If the first light irradiation unit 1001 and the second light irradiation unit 1002 are inclined at the angle 813 and the angle 112 specified by the holding unit 1003 respectively, the area inside the breast 100 where the light propagates is shifted away from the chest wall 101. This is identical to the features as described in Embodiment 1 and Embodiment 2. In this embodiment, the holding unit 1003 includes both the first holding unit and the second holding unit, but the holding unit 1003 may be used only for the first holding unit and another composing element may be used for the second holding unit. For example, if both the first and second holding units are disposed at mutually opposite sides across the breast, a possible interference of the second light irradiation unit, by being located in an area for the first light irradiation unit to irradiate light, can be prevented.

The optical coefficient calculation unit 1004 has the function of the optical coefficient calculation unit 111 in Embodiment 1, and the function to output the calculated average optical coefficient to the light intensity distribution calculation unit 1005.

The light intensity distribution calculation unit 1005 calculates the light intensity distribution inside the breast 100 using the average optical coefficient output from the optical coefficient calculation unit 1004. The calculation method of the light intensity distribution is the same as the light intensity distribution calculation unit 811 in Embodiment 2.

(Effect)

The effect of Embodiment 3 will be described. Since the first light irradiation unit 1001 is inclined at the angle 813, a drop in the initial sound pressure P (r) due to the influence of the chest wall 101 can be reduced for the same reason as Embodiment 2. Further, since the second light irradiation unit 1002 is inclined at the angle 112, the average optical coefficient of the breast 100, influenced less by the chest wall 101, can be acquired for the same reason as Embodiment 1. The light intensity distribution calculation unit 1005 uses the average optical coefficient output from the optical coefficient calculation unit, therefore the light intensity distribution, influenced less by the chest wall 101, can be calculated. This means that the absorption coefficient distribution calculation unit 812 calculates the absorption coefficient distribution μa (r) based on Expression (1), using the initial sound pressure distribution P (r) and the light intensity distribution φ (r), which are both influenced less by the chest wall 101. In other words, the absorption coefficient distribution, influenced less by the chest wall 101, can be acquired.

In the calculation of the absorption coefficient μa (r) of the system in FIG. 5 according to Embodiment 3, the calculation accuracy of the absorption coefficient μa (r) inside the breast 504 is improved.

(Measurement Flow)

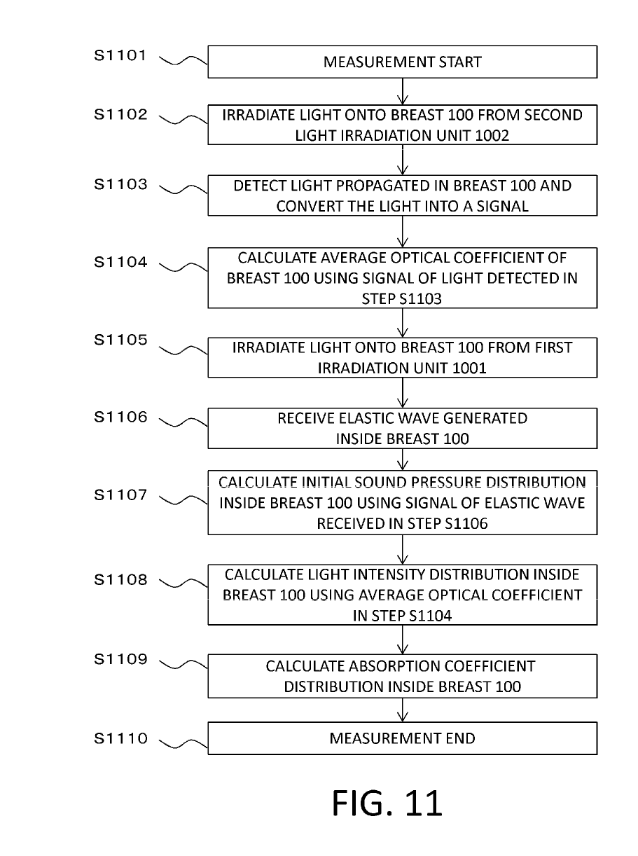

The measurement flow executed by the control unit 115 in Embodiment 3 will be described with reference to FIG. 11.

In step S1101, the measurement starts.

In step S1102, the light generated in the second light source 1007 is guided to the second light irradiation unit 1002 by the irradiation light guiding unit 107, and is irradiated onto the breast 100 held by the holding unit 1003 and the placement unit 807 from the second light irradiation unit 1002.

In step S1103, the light detection unit 103 detects the light which was irradiated onto the breast 100 in step S1102, and is propagated inside the breast 100. The light detected by the light detection unit 103 is guided to the photodetector 110 by the detected light guiding unit 108. The photodetector 110 converts the detected light into a signal. To improve the SN of the signal, the signals may be integrated by repeating step S1102 and step S1103.

In step S1104, the optical coefficient calculation unit 1004 calculates the average optical coefficient of the breast 100 using the signal acquired in step S1103.

In step S1105, the light generated in the first light source 1006 is guided to the first light irradiation unit 1001 by the irradiated light guiding unit 802, and is irradiated from the first light irradiation unit 1001 onto the breast 100.

In step S1106, the transducer array 808 receives an elastic wave generated by the light, which was irradiated onto and propagated inside the breast 100 in step S1105 and absorbed by a light absorber (e.g. cancer) inside the breast 100, and converts the elastic wave into a signal. To improve the SN of the signal, the signals may be integrated by repeating step S1105 and step S1106.

In step S1107, the reconstruction unit 809 calculates the initial sound pressure distribution inside the breast 100 using the signal of the elastic wave received in step S1106.

In step S1108, the light intensity distribution calculation unit 1005 calculates the light intensity distribution inside the breast 100 using the average optical coefficient of the breast 100 calculated in step S1104.

In step S1109, the absorption coefficient distribution calculation unit 812 calculates the absorption coefficient distribution inside the breast 100 using the initial sound pressure distribution calculated in step S1107 and the light intensity distribution calculated in step S1108.

In step S1110, the measurement ends.

As described above, according to Embodiment 3, the absorption coefficient distribution inside the breast 100 can be imaged with high precision by reducing the influence of the chest wall on both the initial sound pressure distribution inside the breast 100 and the average optical coefficient of the breast 100.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-078827, filed on Apr. 4, 2013, which is hereby incorporated by reference herein in its entirety.

What is claimed is:

1. An object information acquiring apparatus, comprising:
an irradiation unit configured to irradiate light from a light source onto a breast, which is an object;
a detection unit configured to detect a signal propagating from the breast by the light irradiated from the irradiation unit;
a calculation unit configured to calculate an optical characteristic inside the breast based on the signal detected by the detection unit, wherein
the irradiation unit configured to irradiate the light onto the breast in a direction away from a chest wall located at the base of the breast.

2. The object information acquiring apparatus according to claim 1, further comprising a holding unit configured to hold the irradiation unit and to be able to fix an irradiation angle, which is an angle of the light irradiated from the irradiation unit.

3. The object information acquiring apparatus according to claim 2, wherein
the holding unit has a plane portion which is approximately parallel with a plane including the chest wall, and the irradiation angle is specified with respect to the plane portion.

4. The object information acquiring apparatus according to claim 2, wherein
the irradiation unit is an optical fiber end.

5. The object information acquiring apparatus according to claim 2, wherein
the holding unit can change the irradiation angle when the irradiation unit is fixed.

6. The object information acquiring apparatus according to claim 2, wherein
the chest wall is a substantially flat plane segment that includes a body of sternum, costal cartilages and pectoralis major muscle of a testee and has an absorption coefficient which is different from that of the breast.

7. The object information acquiring apparatus according to claim 2, wherein
the detection unit detects, as the signal, an elastic wave which is generated inside the breast by the light irradiated from the irradiation unit, and
the calculation unit calculates the absorption coefficient distribution inside the breast as the optical characteristic.

8. The object information acquiring apparatus according to claim 7, further comprising a setting unit that sets an optical coefficient inside the breast, wherein
the calculation unit calculates an initial sound pressure distribution inside the breast, using the elastic wave which is detected,
calculates a light intensity distribution inside the breast, using the optical coefficient which is set, and
calculates the absorption coefficient distribution by correcting the initial sound pressure distribution, using the light intensity distribution.

9. The object information acquiring apparatus according to claim 8, wherein
the setting unit sets the optical coefficient using an input value from an operator.

10. The object information acquiring apparatus according to claim 8, further comprising:
a second irradiation unit configured to irradiate light from a second light source onto the breast; and
a second detection unit configured to detect the light propagated inside the beast after being irradiated from the second irradiation unit, wherein
the calculation unit calculates an optical coefficient inside the breast based on the light intensity detected by the second detection unit, and
the setting unit sets the calculated value as the optical coefficient used for calculating the light intensity distribution.

11. The object information acquiring apparatus according to claim 10, wherein
the holding unit holds the second irradiation unit so as to irradiate light onto the breast in a direction away from the chest wall.

12. The object information acquiring apparatus according to claim 2, wherein
the detection unit detects, as the signal, the light propagated inside the breast after being irradiated from the irradiation unit, and
the calculation unit calculates the optical coefficient inside the breast as the optical characteristic.

13. The object information acquiring apparatus according to claim 12, wherein
the detection unit is fixed by the holding unit so as to detect light at an angle according to the irradiation angle.

14. The object information acquiring apparatus according to claim 12, wherein
the calculation unit calculates, as the optical coefficient, at least one of an absorption coefficient, a scattering coefficient and an effective attenuation coefficient.

15. A control method for an object information acquiring apparatus having an irradiation unit, a detection unit and a calculation unit,
the method comprising:
an irradiation step of operating the irradiation unit to irradiate light from a light source onto a breast, which is an object;
a detection step of operating the detection unit to detect a signal propagating from the breast by the light irradiated from the irradiation unit; and
a calculation step of operating the calculation unit to calculate an optical characteristic inside the breast based on the signal detected by the detection unit, wherein
in the irradiation step, the irradiation unit irradiates light onto the breast in a direction away from the chest wall located at the base of the breast.
